(19) United States
(12) Patent Application Publication
(10) Pub. No.: US 2015/0032124 A1
(43) Pub. Date: Jan. 29, 2015

(54) Cochlear Implant Electrode Insertion Support Device

(71) Applicant: MED-EL Elektromedizinische Geraete GmbH, Innsbruck (AT)

(72) Inventors: Thomas Lenarz, Hannover (DE); Ingeborg Hochmair, Axams (AT); Claude Jolly, Innsbruck (AT); Anandhan Dhanasingh, Innsbruck (AT); Andreas Harnisch, Innsbruck (AT)

(21) Appl. No.: 14/334,813

(22) Filed: Jul. 18, 2014

Related U.S. Application Data
(60) Provisional application No. 61/858,659, filed on Jul. 26, 2013.

(51) Int. Cl. A61B 17/34; A61N 1/05

(52) U.S. Cl. CPC A61B 17/3468; A61N 1/0541
USPC 606/129

(57) ABSTRACT

An electrode insertion support device is used for inserting a cochlear implant electrode into a cochlea scala of a patient cochlea. A stiff electrode holder encloses at least a portion of a cochlear implant electrode while allowing the electrode within to slide freely. A pointed distal tip of the electrode holder is adapted to pierce an electrode opening through an outer surface of the patient cochlea into the cochlea scala. The insertion support device prevents an apical tip of the enclosed electrode from contacting tissues around the electrode opening during the insertion surgery.

This application claims priority from U.S. Provisional Patent Application 61/858,659, filed Jul. 26, 2013, which is incorporated herein by reference in its entirety.

### FIELD OF THE INVENTION

The present invention relates to an insertion device for cochlear implant electrodes.

### BACKGROUND ART

A normal ear transmits sounds as shown in FIG. 1 through the outer ear 101 to the tympanic membrane 102 which moves the bones of the middle ear 103 that vibrate the oval window and round window openings of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

Hearing is impaired when there are problems in the ability to transduce external sounds into meaningful action potentials along the neural substrate of the cochlea 104. To improve impaired hearing, auditory prostheses have been developed. For example, when the impairment is related to operation of the middle ear 103, a conventional hearing aid may be used to provide acoustic-mechanical stimulation to the auditory system in the form of amplified sound. Or when the impairment is associated with the cochlea 104, a cochlear implant with an implanted electrode can electrically stimulate auditory nerve tissue with small currents delivered by multiple electrode contacts distributed along the electrode.

FIG. 1 also shows some components of a typical cochlear implant system where an external microphone provides an audio signal input to an external signal processor 111 in which various signal processing schemes can be implemented. The processed signal is then converted into a digital data format for transmission by external transmitter coil 107 into the implant 108. Besides receiving the processed audio information, the implant 108 also performs additional signal processing such as error correction, pulse formation, etc., and produces a stimulation pattern (based on the extracted audio information) that is sent through an electrode lead 109 to an implanted electrode array 110. Typically, this electrode array 110 includes multiple stimulation contacts 112 on its surface that provide selective stimulation of the cochlea 104.

The electrode array 110 contains multiple electrode wires embedded in a soft silicone body referred to as the electrode carrier. The electrode array 110 needs to be mechanically robust, and yet flexible and of small size to be inserted into the cochlea 104. The material of the electrode array 110 needs to be soft and flexible in order to minimize trauma to neural structures of the cochlea 104. But an electrode array 110 that is too floppy tends to buckle too easily so that the electrode array 110 cannot be inserted into the cochlea 104 up to the desired insertion depth. A trade-off needs to be made between a certain stiffness of the electrode array 110 which allows insertion into the cochlea 104 up to the desired insertion depth without the array buckling, and certain flexibility of the electrode array 110 which keeps mechanical forces on the structures of the scala tympani of the cochlea 104 low enough.

One of the important steps in cochlear implant surgery is the insertion of the electrode array into the scala tympani of the cochlea. Starting from the opening of the electrode opening in the round window membrane until complete full insertion of the electrode array 110, the insertion surgery should be as atraumatic as possible in order to preserve residual hearing. To achieve that goal, the surgical opening of the round window membrane and the electrode insertion technique should be a uniform reproducible procedure. But the reality is that some surgeons cut a slit in the round window membrane, while others create a flap opening.

In some cases, the electrode insertion process can be disrupted when the flexible tip of the electrode array 110 slips and sticks to the wet tissues around the electrode opening into the cochlea 104 rather than entering through the electrode opening as desired. This is frustrating and time consuming for the surgeon who often has to make repeated efforts to thread the tip of the electrode array 110 through the electrode opening. In addition, the contact with the wet tissues can deposit blood and other fluids onto the tip of the electrode array 110 which then contaminate the interior of the cochlea 104. Surgeons also try to insert the electrode array 110 as slowly as possible by step-wise advancing it into the scala tympani which also is time consuming.

### SUMMARY OF THE INVENTION

Embodiments of the present invention are directed to an electrode insertion support device and corresponding surgical method for inserting a cochlear implant electrode into a cochlea scala of a patient cochlea. A stiff electrode holder encloses at least a portion of a cochlear implant electrode while allowing the electrode within to slide freely. A pointed distal tip of the electrode holder is adapted to pierce an electrode opening through an outer surface of the patient cochlea into the cochlea scala. The insertion support device prevents an apical tip of the enclosed electrode from contacting tissues around the electrode opening during the insertion surgery.

There may be a flexible electrode cover around at least a portion of the electrode holder adapted to allow placement of the electrode into the electrode holder. There may also be a device handling feature at a proximal end of the electrode holder for surgical manipulation of the insertion support device and the enclosed electrode during the insertion surgery. And there may be an insertion stopper near the distal tip adapted to limit penetration distance of the distal tip into the cochlea scala. An electrode projection mechanism may allow pushing the enclosed electrode through the electrode opening into the cochlea scala.

In specific embodiments, the electrode holder may be cylindrical and may form an open tubular section enclosing the at least a portion of the cochlear implant electrode, and/or a perforated tubular section enclosing the at least a portion of the cochlear implant electrode. The electrode holder includes an inner surface coated with a reduced friction material that promotes free sliding of the enclosed portion of the cochlear implant electrode.

The electrode holder may be sized to fit within a mastoidectomy passage during the insertion surgery; for example, there may be a mastoidectomy section sized to fit within a mastoidectomy passage and tympanotomy section sized to fit within a posterior tympanotomy, wherein the two sections meet at a connecting angle that changes the direction of the enclosed electrode.

### BRIEF DESCRIPTION OF THE DRAWINGS

FIG. 1 shows the anatomy of the human ear with a cochlear implant system.

FIG. 2 shows an example of an electrode insertion support device according to one embodiment of the present invention.

FIG. 3 shows structural details of the distal tip of an electrode insertion support device.

FIG. 4 highlights the sharpness of the distal tip.

FIG. 5 shows an alternative embodiment of an electrode cover having a handling feature.

FIG. 6 shows an alternative embodiment having a mastoidectomy section sized to fit within a mastoidectomy passage and tympanotomy section sized to fit within a posterior tympanotomy with a connecting angle that changes the direction of the enclosed electrode.

FIG. 7 shows an alternative embodiment having a perforated tubular section.

FIG. 8 A-D shows details of cochlear implant electrodes having electrode projection features that allow pushing the electrode through the electrode opening into the cochlea scala.

FIG. 9 shows an electrode insertion support device with a cochlear implant electrode enclosed within for insertion into a patient cochlea.

FIG. 10 shows an example of a surgical insertion tool incorporating a an electrode insertion support device according to an embodiment of the present invention.

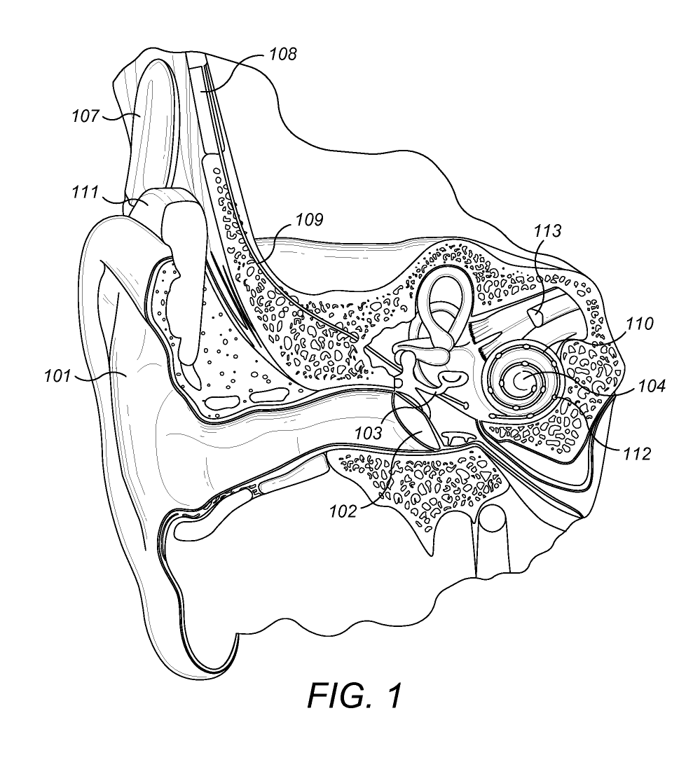

### DETAILED DESCRIPTION OF SPECIFIC EMBODIMENTS

Embodiments of the present invention are based on using an electrode insertion support device that promotes a uniform incision of the electrode opening (e.g., in the round window membrane) and a uniform electrode insertion procedure which avoids the electrode array contacting and being contaminated by the surrounding tissue.

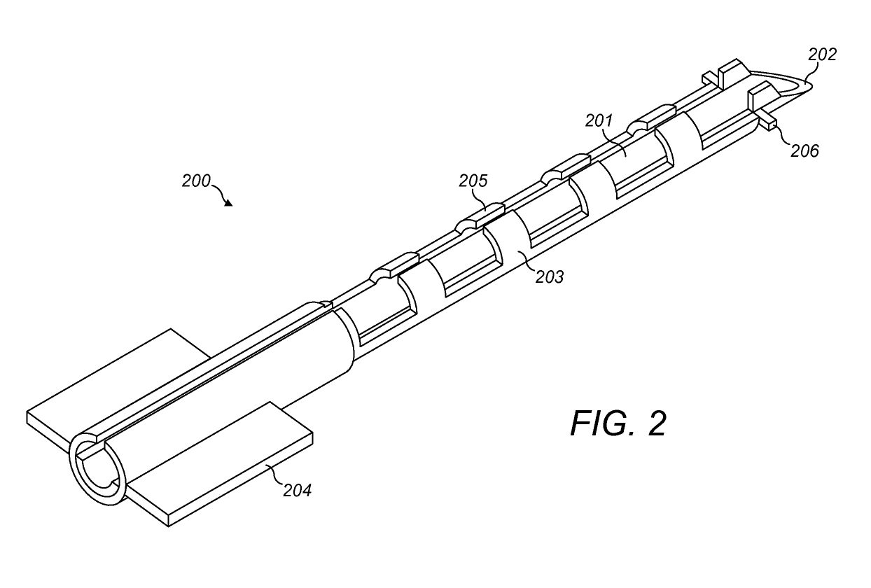

FIG. 2 shows an electrode insertion support device 200 which includes a stiff electrode holder 201 made of any structurally appropriate biocompatible material (e.g., made of metal or stiff plastic) that encloses at least a portion of a cochlear implant electrode while allowing the enclosed electrode to slide freely within. The electrode holder 201 may include an inner surface 207 coated with a reduced friction material that promotes free sliding of the enclosed portion of the cochlear implant electrode; for example, a highly hydrophilic material such as a hydrogel. In addition or alternatively, the inner surface 207 may be coated with a special coating that generally absorbs liquids (again, e.g., a hydrogel) so that even if some blood is present in the middle ear during insertion surgery and enters within the electrode holder 201, that blood will absorbed by the coating material on the inner surface 207 and will not enter the cochlea together with the electrode.

A pointed distal tip 202 of the electrode holder 201 is adapted to pierce an electrode opening through an outer surface of the patient cochlea into the cochlea scala. The insertion support device 200 prevents an apical tip of the enclosed electrode from contacting tissues around the electrode opening during the insertion surgery.

The embodiment depicted in FIG. 2 includes a flexible electrode cover 203 around at least a portion of the electrode holder 201 which adapted to allow placement of the electrode into the electrode holder 201. In this example, the electrode cover 203 includes multiple flexible slit ribs 205 that easily deflect aside for insertion of the implant electrode into the open tubular section (i.e., half-pipe) of the electrode holder 201. At the proximal end of the electrode holder 201 is a device handling feature, in this case a wing projection 204, for surgical manipulation and orientation of the insertion support device 200 and the enclosed electrode during the insertion surgery.

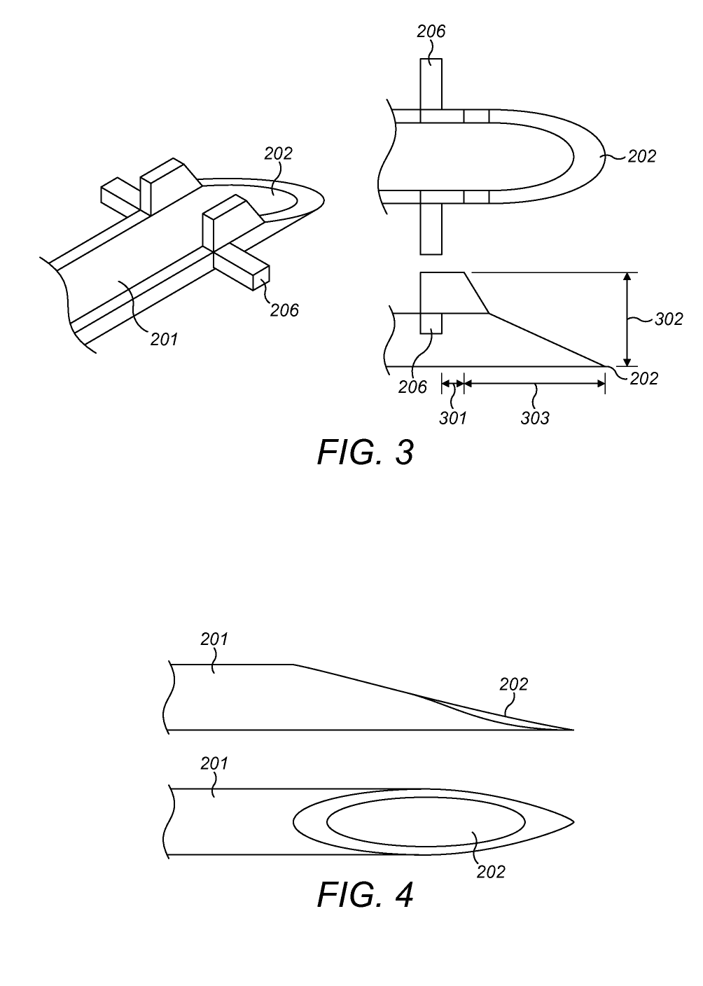

FIG. 3 shows structural details of the distal tip 202 of the electrode insertion support device 200 and FIG. 4 highlights the sharpness of the distal tip 202. An insertion stopper 206 is located near the distal tip 202 to limit penetration distance of the distal tip 202 into the cochlea scala. Specifically, the distal tip 202 typically has a length of not more than about one millimeter which is the desired insertion depth of the electrode insertion support device 200 inside the scala tympani. The height 302 of the distal tip 202 is important in controlling a proper incision of the electrode opening. In particular, the height 302 should be about the same as or slightly greater than the thickness of the electrode array 110 at its basal end. The distal tip 202 has a sharp edge (see FIG. 4) all the way along its cutting length 303 which enables a rotational movement of the distal end 202 to open/incise the electrode opening.

Immediately after the cutting edge of the distal tip 202 is a short flat section 301 without a cutting edge before reaching an insertion stopper 206 that prevents over-insertion of the distal tip 202 into the scala tympani. The dimensions of the flat section 301 (e.g. 50 μm) and the insertion stopper 206 allow the distal tip 202 to be inserted far enough to overcome the elastic character of the round window membrane to promote an optimal incision of the electrode opening, preferably creating a flap from a portion of the round window membrane rather than a relatively large hole as would be the case with the tip of a standard syringe.

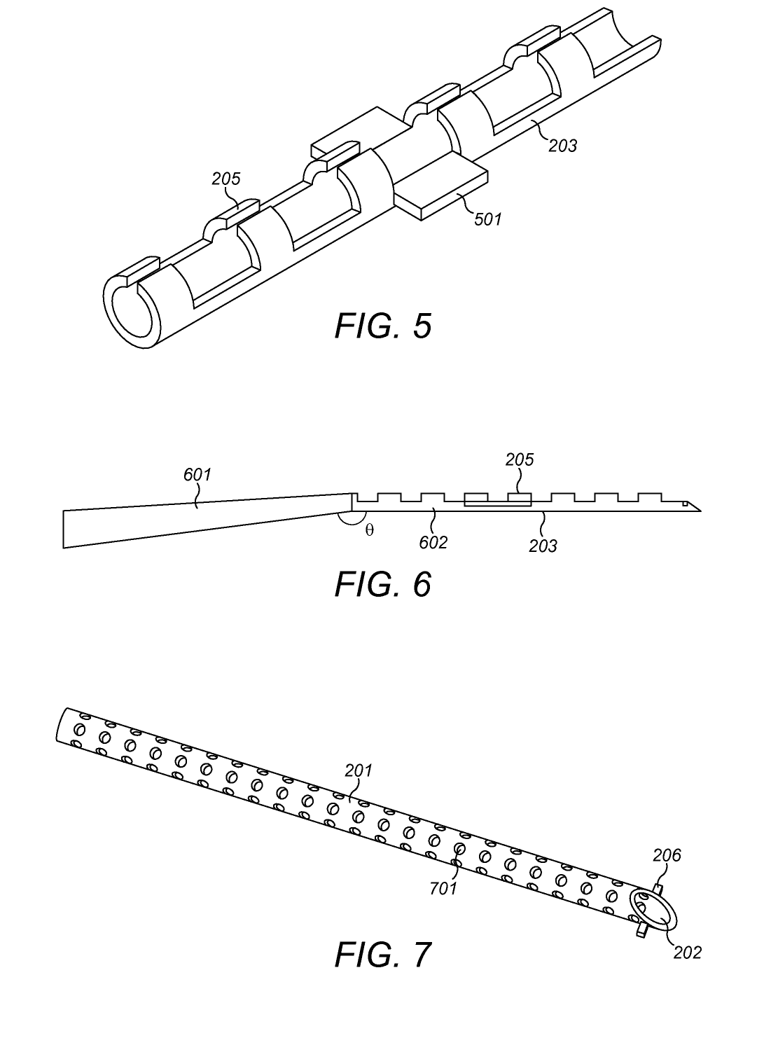

FIG. 5 shows an alternative embodiment of an electrode cover 203 with slit ribs 205 and handling wing 501 located in the middle of the electrode cover 203 for surgical handling and orientation of an electrode insertion support device 200. Locating the handling wing 501 in the middle of the electrode cover 203 as shown allows the handling wing 501 to conveniently be positioned in the facial recess of the posterior tympanotomy after insertion of the electrode array 110 into the cochlea so that the electrode insertion support device 200 can easily be removed.

The relative geometries of the mastoidectomy and the tympanotomy are such that it may be useful to have an angle between the relative sections of the electrode insertion support device 200 that are intended to fit into those spaces. For example, FIG. 6 shows an embodiment of an electrode insertion support device 200 having a mastoidectomy section 601 sized to fit within a mastoidectomy passage and tympanotomy section 602 sized to fit within a posterior tympanotomy with a connecting angle θ that changes the direction of the enclosed electrode array 110. The electrode insertion support device 200 and the electrode array 110 enclosed within are gently introduced into the posterior tympanotomy where the sharp tip 202 of the electrode insertion support device 200 can incise the electrode opening in the round window membrane.

FIG. 7 shows an alternative embodiment of an electrode insertion support device 200 wherein the electrode holder 201 includes a perforated tubular section 701. With a fully enclosed tubular section 701 it may not be possible to remove the electrode insertion support device 200 after surgical insertion of the electrode array 110 into the cochlea. So such an embodiment would need to be suitable for permanent implantation inside the skull.

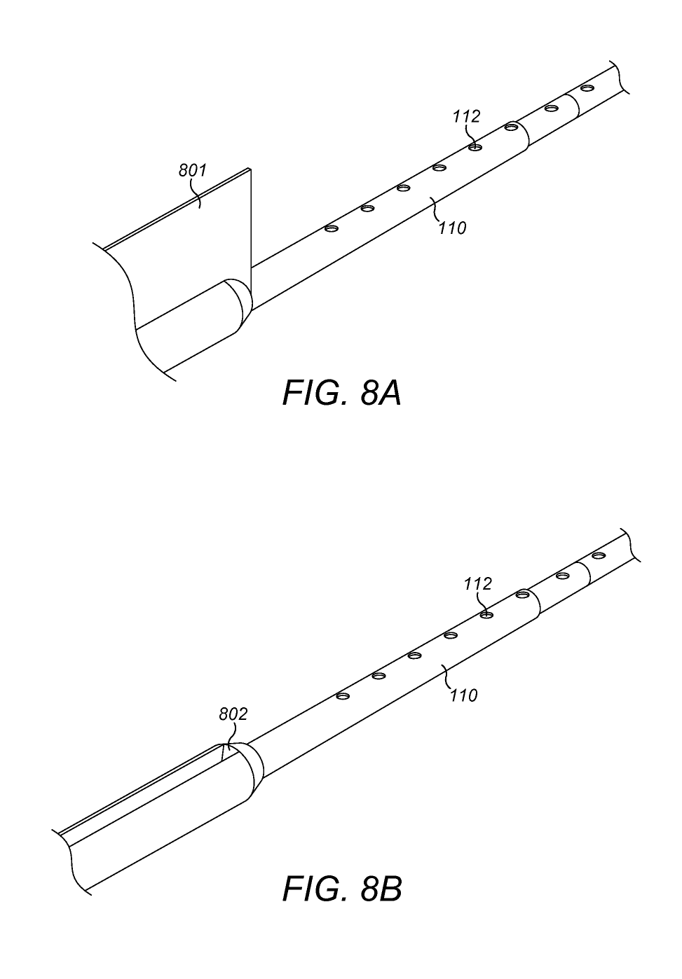

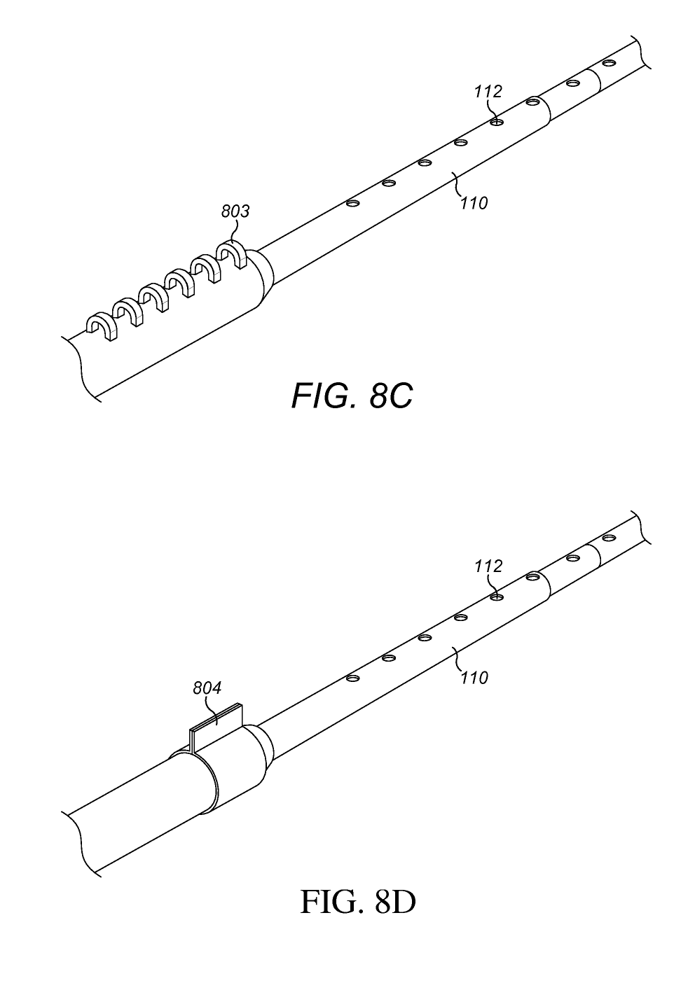

For ease in inserting the electrode array 110 into the cochlea, it may be useful for the combination of the electrode insertion support device 200 and the electrode array 110 to include one or more electrode projection mechanisms. FIG. 8 A-D shows details of electrode arrays 110 having electrode projection mechanisms that allow pushing the electrode array 110 through the electrode opening into the cochlea scala. FIG. 8A shows an example of a pushing wing 801 that allows the electrode array 110 to be pushed into the scala tympani during insertion surgery. FIG. 8B an electrode array 110 having a projection disk 802 at its base that coordinates with a groove in the electrode insertion support device 200 to allow pushing of the electrode array 110. In FIG. 8C, the base of the electrode array 110 includes one or more handling rings 803 that allow pushing of the electrode array 110 into the scala tympani. And as shown in FIG. 8D, an electrode projection mechanism may be based on a temporary handling clip 804 that fits onto the electrode array 110 for use during the insertion surgery, and then be removed when the electrode insertion support device 200 is removed.

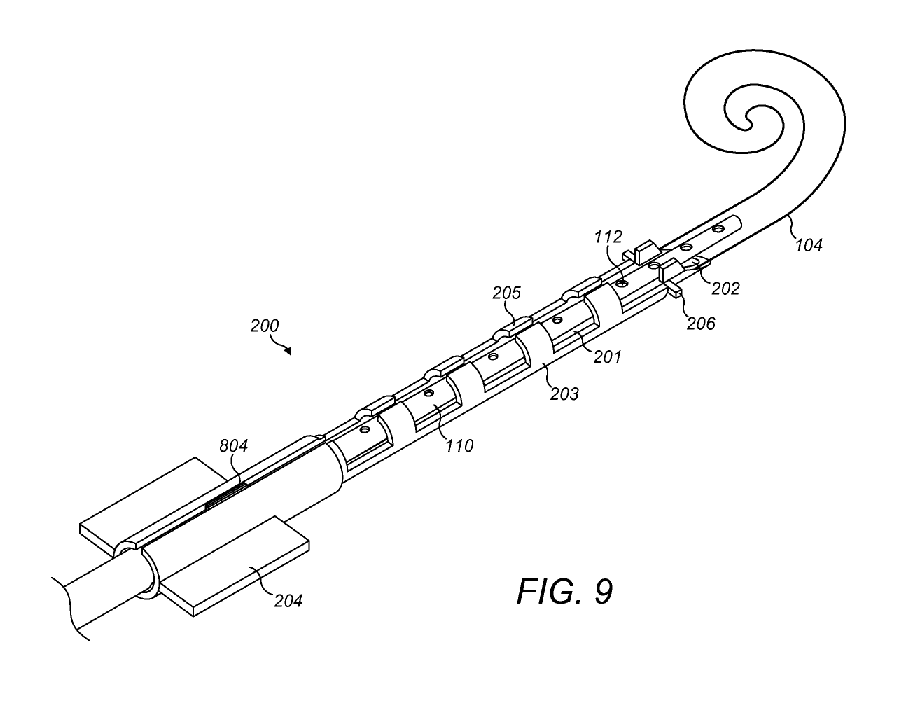

FIG. 9 shows an electrode insertion support device 200 with an electrode array 110 enclosed within for insertion into a patient cochlea 104. The sharp distal tip 202 pierces the round window membrane and just barely enters into the scala tympani. The electrode array 110 within the electrode insertion support device 200 can then be pushed into the cochlea 104 without contacting the surrounding tissues.

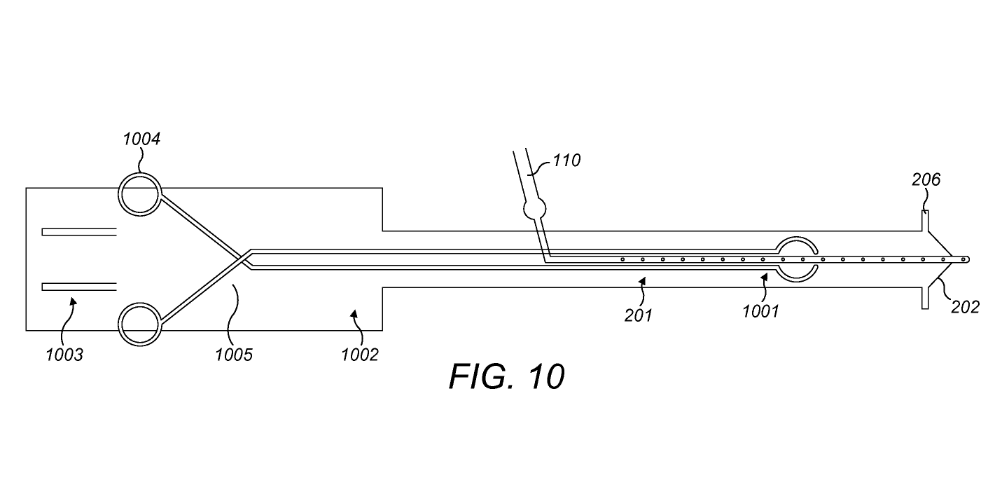

FIG. 10 shows an example of a surgical insertion tool 1001 incorporating an electrode holder 201 according to an embodiment of the present invention. The tool base 1002 may include one or more sliding grooves 1003 on which the surgical insertion tool 1001 is mounted. The electrode holder 201 is also mounted on the tool base 1002. The insertion tool 1001 has handles 1004 that allow the insertion tool 1001 to control the electrode array 110 enclosed within the electrode holder 201 to push it into the cochlea.

Although various exemplary embodiments of the invention have been disclosed, it should be apparent to those skilled in the art that various changes and modifications can be made which will achieve some of the advantages of the invention without departing from the true scope of the invention.

What is claimed is:

1. An electrode insertion support device for inserting a cochlear implant electrode into a cochlea scala of a patient cochlea, the insertion support device comprising:
a stiff electrode holder enclosing at least a portion of a cochlear implant electrode while allowing the electrode within to slide freely; and
a pointed distal tip of the electrode holder adapted to pierce an electrode opening through an outer surface of the patient cochlea into the cochlea scala;
wherein the insertion support device prevents an apical tip of the enclosed electrode from contacting tissues around the electrode opening during the insertion surgery.

2. An electrode insertion support device according to claim 1, further comprising:
a flexible electrode cover around at least a portion of the electrode holder adapted to allow placement of the electrode into the electrode holder.

3. An electrode insertion support device according to claim 1, further comprising:
a device handling feature at a proximal end of the electrode holder for surgical manipulation of the insertion support device and the enclosed electrode during the insertion surgery.

4. An electrode insertion support device according to claim 1, further comprising:
an insertion stopper near the distal tip adapted to limit penetration distance of the distal tip into the cochlea scala.

5. An electrode insertion support device according to claim 1, wherein the implant electrode includes an electrode projection mechanism for pushing the electrode through the electrode opening into the cochlea scala.

6. An electrode insertion support device according to claim 1, wherein the electrode holder forms an open tubular section enclosing the at least a portion of the cochlear implant electrode.

7. An electrode insertion support device according to claim 1, wherein the electrode holder forms a perforated tubular section enclosing the at least a portion of the cochlear implant electrode.

8. An electrode insertion support device according to claim 1, wherein the electrode holder includes a mastoidectomy section sized to fit within a mastoidectomy passage and tympanotomy section sized to fit within a posterior tympanotomy, wherein the two sections meet at a connecting angle that changes the direction of the enclosed electrode.

9. An electrode insertion support device according to claim 1, wherein the electrode holder is sized to fit within a mastoidectomy passage during the insertion surgery.

10. An electrode insertion support device according to claim 1, wherein the electrode holder includes an inner surface coated with a reduced friction material that promotes free sliding of the enclosed portion of the cochlear implant electrode.

11. A method for inserting a cochlear implant electrode into a cochlea scala of a patient cochlea, the method comprising:
enclosing at least a portion of a cochlear implant electrode within a stiff electrode holder while allowing the electrode within to slide freely while preventing an apical tip of the enclosed electrode from contacting tissues around the electrode opening during the insertion surgery;
piercing an electrode opening through an outer surface of the patient cochlea into the cochlea scala with a pointed distal tip of the electrode holder; and
inserting the electrode over the apical tip and into the cochlea scala.

12. A method according to claim 11, further comprising:
providing a flexible electrode cover around at least a portion of the electrode holder which is adapted to allow placement of the electrode into the electrode holder.

13. A method according to claim 11, a device handling feature at a proximal end of the electrode holder is used for surgical manipulation of the insertion support device and the enclosed electrode when inserting the electrode.

14. A method according to claim 11, wherein an insertion stopper near the distal tip limits penetration distance of the distal tip into the cochlea scala when piercing an electrode opening.

15. A method according to claim 11, wherein the implant electrode includes an electrode projection mechanism for pushing the electrode through the electrode opening into the cochlea scala.

16. A method according to claim 11, wherein the electrode holder forms an open tubular section enclosing the at least a portion of the cochlear implant electrode.

17. A method according to claim 11, wherein the electrode holder forms a perforated tubular section enclosing the at least a portion of the cochlear implant electrode.

18. A method according to claim 11, wherein the electrode holder includes a mastoidectomy section sized to fit within a mastoidectomy passage and tympanotomy section sized to fit within a posterior tympanotomy, wherein the two sections meet at a connecting angle that changes the direction of the enclosed electrode.

19. A method according to claim 11, wherein the electrode holder is sized to fit within a mastoidectomy passage during the insertion surgery.

20. A method according to claim 11, wherein the electrode holder wherein the electrode holder includes an inner surface coated with a reduced friction material that promotes free sliding of the enclosed portion of the cochlear implant electrode.
